(19) United States
(12) Patent Application Publication
(10) Pub. No.: US 2002/0006433 A1
(43) Pub. Date: Jan. 17, 2002

(54) PHARMACEUTICAL FORMULATIONS

(76) Inventors: NIGEL P. DAVIDSON, BRISTOL, TN (US); CREIGHTON P. CONLEY, BRISTOL, TN (US)

Correspondence Address: GLAXOSMITHKLINE, CORPORATE INTELLECTUAL PROPERTY-UW2220, P.O. BOX 1539, KING OF PRUSSIA, PA, 19406-0939, US

(21) Appl. No.: 09/301,838

(22) Filed: Apr. 29, 1999

(51) Int. Cl. A61K 47/00; A61K 9/68; A61K 9/28; A61K 9/14

(52) U.S. Cl. 424/441; 424/439; 424/489

(57) ABSTRACT

A chewable tablet comprising amoxycillin in which the chewable base comprises essentially mannitol which is present in at least 25% by weight of the tablet are useful for treating a wide range of bacterial infections in children.

### FIELD OF THE INVENTION

The present invention relates to novel pharmaceutical formulations comprising amoxycillin.

### BACKGROUND OF THE INVENTION

Amoxycillin is an anti-bacterial agent extensively used for the treatment of a wide range of bacterial infections. It is currently available in a number of different formulations, for instance, as capsules, chewable tablets, oral suspensions and paediatric drops. Different formulations and different amounts of amoxycillin are provided for adult and paediatric patients. Thus, for example, SmithKline Beecham market, under the trade mark “Amoxil”, inter alia chewable tablets comprising either 125 mg or 250 mg amoxycillin, for adult patients, and, for paeditric patients, an oral suspension comprising either 125 mg or 250 mg/5 ml of suspension. Existing formulations are approved for dosing at least three times a day (tid). These chewable tablets contain a substantial amount of sugar (about 21% by weight) which makes the tablet base less suited for adaption to a paediatric tablet. In addition, SmithKline Beecham also market, under the trade mark “Augmentin”, inter alia chewable tablets comprising 125 mg amoxycillin and 31.25 mg potassium clavulanate or 250 mg amoxycillin and 62.5 mg potassium clavulanate, for administration to adult patients.

EP 0 396 335-A1 (Beecham Group plc) describes chewable effervescent tablets, for instance a “fizzy chewable” tablet comprising amoxycillin (250 mg) in which the chewable base comprises a mixture of sorbitol and mannitol, present in about 15% and 31% by weight of the tablet, respectively. The effervescent couple helps mask the unpleasant bitter taste of amoxycillin. A disintegrant such as a cellulose product may also be present, in an amount from 5 to 30%, to give the patient the option of dispersing the tablet in a small amount of water prior to administration.

WO 92/19227 (Laboratorios Beecham SA) describes swallow tablets comprising amoxycillin which are prepared from granules formed by roller compaction of amoxycillin with a disintegrant.

There still remains a need to provide further formulations comprising amoxycillin, which provide an alternative to existing formulations and which may also provide for a more convenient dosage regimen, especially for children.

### SUMMARY OF THE INVENTION

The present invention provides for a chewable tablet comprising amoxycillin trihydrate in which the chewable base comprises essentially mannitol which is present in at least 25% by weight of the tablet.

### DETAILED DESCRIPTION OF THE INVENTION

The present invention provides for a chewable tablet preferably adapted for administration to a paediatric patient.

The tablet is also preferably provided for in both a low amount dosage form and a high amount dosage form. Conventionally, the amount of amoxycillin trihydrate present in a dosage form is expressed as the weight of the corresponding free acid amoxycillin. Preferably, the low dosage form comprises from about 150 to 250 mg, more preferably 175 to 225 mg, typically about 200 mg amoxycillin. Preferably, the high dosage form comprises about 350 to 450, more preferably 375 to 425 mg, typically about 400 mg amoxycillin.

Preferably, the amoxycillin trihydrate is present in either dosage form from about 45 to about 60%, more preferably from about 50 to about 55% by weight of the tablet.

Preferably, mannitol is present from about 30 toabout 50%, more preferably from about 35 to about 45%, yet more preferably from about 40 to sbout 45%, by weight of the tablet. Preferably, a granular grade of mannitol is used.

For manufacturing convenience, it is preferred to maintain a constant tablet weight. Preferably, the amount of mannitol is adjusted to compensate for any variation in the amount of amoxycillin trihydrate, on account of the purity thereof. Preferably, amoxycillin trihydrate and mannitol together comprise from about 90 to about 97%, more preferably about 92 about 96%, yet more preferably about 93 to about 96% by weight of the tablet.

In addition to an excipient to provide a chewable base, a tablet according to the present invention may also comprise further excipients, for instance, disintegrants, lubricants, sweetening agents, colouring and flavouring agents. Such further excipients together will preferably comprise from 3 to 10%, more preferably 4 to 8%, yet more preferably 4 to 7% by weight of the tablet.

Disintegrants may be present in from 1 to 4%, preferably from 1 to 3%, more preferably from 1 to 2% by weight of the tablet. Representative disintegrants include crospovidone, sodium starch glycollate, starches such as maize starch and rice strach, croscarmellose sodium and cellulose products such as microcrystalline cellulose, microfine cellulose, low substituted hydroxy propyl cellulose, either used singly or in admixture. Preferably, the disintegrant is crospovidone. The presence of a disintegrant such as crospovidone in the tablet is found to produce a dramatically faster dissolution rate.

Lubricants may be present in from 0.25 to 2.0%, preferably from 0.5 to 1.2% by weight of the tablet. Preferred lubricants include magnesium stearate.

Preferably, the sweetening agent is an artificial sweetening agent such as sodium saccharin or aspartame, preferably aspartame, which may be present in from 0.5 to 1.5% by weight of the tablet. Preferably, a tablet of the present invention is substantially free of sugar (sucrose).

Preferred flavouring agents include fruit flavours which may be natural or synthetic, for instance peppermint, cherry and banana, or a mixture thereof. Preferably, a tablet of the present invention is substantially free of a flavour enhancer such as glycine.

Preferably, the low dosage tablet has a total weight in the range 400 to 500 mg, more preferably 425 to 475 mg, typically about 450 mg. Preferably, the high dosage tablet has a total weight in the range 850 to 950 mg, more preferably 875 to 925 mg, typically about 900 mg.

In a prefered embodiment, a tablet of the present invention comprises from 50 to 55% amoxycillin trihydrate; from 40 to 45% mannitol; from 1 to 2% disintegrant, preferably, crospovidone; from 0.5 to 1.2% lubricant, preferably magnesium stearate; from 0.5 to 1.5% artificial sweetening agent, preferably aspartame; and flavouring and colouring agents, the % being expressed as % of the weight of the tablet.

Preferably, the chewable tablets of the present invention are used for treating a wide range of bacterial infections in paediatric patients, such as infections of the ear, nose and throat, infections of the genitourinary tract, infections of the lower respiratory tract and skin and soft tissue infections. Preferably, the unit dosage is taken every 12 h (bid, q12 h). This provides a more conveninent dosage regimen for a paediatric patient, as there is no need to dose the child during the middle of the day, when the child may be at school.

Tablets of the present invention may be prepared by blending together the various excipients together with amoxycillin by conventional techniques, for example by direct compression. Alternatively, some of the ingredients may be first slugged or subjected to roller compaction and resultant slugs or flakes then milled to form granules. These granules are then blended with the remaining ingredients and finally compressed into tablets. The preparation of granules comprising amoxycillin trihydrate has previously been described in WO 92/19227 (Laboratorios Beecham SA) and in WO 98/35672 (SmithKline Beecham Laboratoires Pharmaceutiques). In a preferred process, granules are prepared by roller compaction and then milling from a primary blend comprising amoxycillin trihydrate, mannitol, the disintegrant and about 50% of the lubricant. The granules are then blended with the remaining lubricant and any remaining other excipients to form a secondary blend which is then and compressed into tablets. Preferably, the sweetening and colouring agents are incorporated into the primary blend while the flavouring agents are incorporated into the secondary blend.

Granules formed with mannitol are novel. Accordingly, in another aspect the present invention provides for granules comprising amoxycillin trihydrate present in from about 45 to about 60%, more preferably 50 to 55% by weight of the granule and mannitol present in from about 25%, preferably 30 to 50%, more preferably 35 to 45%, yet more preferably 40 to 45%, by weight of the granule.

Preferably, the granules further comprise a disintegrant as hereinbefore described, present in from 1 to 4%, preferably from 1 to 3%, more preferably from 1 to 2% by weight of the granule.

Preferably, the granules further comprise a lubricant as hereinbefore described, present in 0.125 to 1.0%, preferably from 0.25 to 0.6% by weight of the granule.

Prefered granules comprise amoxycillin trihydrate present in from 50 to 55%, mannitol present in from 40 to 45%, crospovidone present in from 1 to 2%, and magnesium stearate present in from 0.25 to 0.6%; all percents (%) being by weight of the granule.

In a further aspect, the present invention provides for a chewable tablet comprising granules as hereinbefore described. Preferably, the tablets comprise an extra-granular lubricant, present in about 50% of the total amount of lubricant.

The chewable tablets are preferably packaged in sealed protective containers, such as screw cap bottles, plastic or metal tubes, aluminium foil sachets, aluminium blister packs, etc.

The invention is illustrated by the following Examples:

| | | |
|---|---|---|
| Amoxycillin trihydrate* | 232.56 mg | 51.68% |
| (equivalent to amoxycillin free acid | 200) | |
| Mannitol (granular) | 190.93 | 42.43 |
| Crospovidone | 7.02 | 1.56 |
| Aspartame | 3.24 | 0.72 |
| Magnesium stearate | 4.50 | 1.00 |
| Colouring agent | 0.27 | 0.06 |
| Flavouring agents | 11.48 | 2.55 |
| Total tablet weight | 450 | |

*based on 86% potency (the “potency” being an adjustment to take account of for the purity and the trihydrate content). The quantity of mannitol is adjusted according to the potency of the amoxycillin trihydrate, in order to maintain a constant tablet weight.

| | | |
|---|---|---|
| Amoxycillin trihydrate* | 465.12 mg | 51.68% |
| (equivalent to amoxycillin free acid | 400) | |
| Mannitol (granular) | 381.87 | 42.43 |
| Crospovidone | 14.04 | 1.56 |
| Aspartame | 6.48 | 0.72 |
| Magnesium stearate | 9.00 | 1.00 |
| Colouring agent | 0.54 | 0.06 |
| Flavouring agents | 22.95 | 2.55 |
| Total tablet weight | 900 | |

*based on 86% potency (the “potency” figure being an adjustment to take account of both the purity and the trihydrate content). The quantity of mannitol is adjusted according to the potency of the amoxycillin trihydrate, in order to maintain a constant tablet weight.

The tablets were prepared by initially blending together amoxycillin trihydrate, crospovidone, colouring agent, aspartame, mannitol and magnesium stearate (½ quantity). The blend was then processed by roller compaction and milled to form granules. These granules were blended with the flavouring agents and the remaining magnesium stearate and the overall blend compressed to form tablets.

### REFERENCE EXAMPLE 1

Existing 250 mg Chewable Tablet (Amoxil)*

Amoxycillin 250 mg (as amox trihydrate)

Inactive ingredients: citric acid, cornstarch*, FD&C Red No. 40, flavorings, glycine, mannitol, magnesium stearate, saccharin sodium, silica gel and sucrose. *Physicians Desk Reference, 49 edn, Medical Economics Data Production Co, Montvale N.J. 07645-1742, 2351, 1995 ** a constituent of the product comprising the flavourings

Dissolution Assay

The method used was that described in the The United States Pharmacopeia (USP 23, 1995) at page 104, for Amoxicillin and Clavulanate Potassium Tablets, adapted as follows:

| Apparatus: | 2 (paddles) |
|---|---|
| RPM: | 75 |
| Media: | 900 ml of 37° C. water |
| For 200 and 400 mg chewable tablets (Examples 1 and 2) | |
| Time | 30 min |
| Q | 75% |
| For 125 and 250 mg chewable tablets (Reference Example): | |
| Time | 90 min |
| Q | 80% |
| For Capsule formulation comprising amoxycillin: | |
| Apparatus: | 1 (baskets) |
| RPM: | 100 |
| Media | 900 ml of 37° C. water |
| Time: | 90 minutes |
| Q = | 80% |

Thus, the presence in the chewable tablets of the present invention of a disintegrant, crospovidone, was found to dramatically increase the dissolution rate of the chewable tablet, compared with an existing chewable tablet (Reference Example 1) which does not have a disintegrant. There was 75% dissolution after 30 minutes, compared with 80% dissolution after 90 minutes. Furthermore, the chewable tablets of the invention had an improved dissolution rate when compared to the existing Amoxil (Trade Mark) capsule formulation.

Bioequivalence Study

In addition, in a bioequivalance study in human volunteers, the 400 mg chewable tablet was demonstrated to be bioequivalent to a paediatric suspension of amoxycillin comprising a corresponding amount of amoxycillin per 5 μl of suspension.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A chewable tablet comprising amoxycillin trihydrate in which the chewable base comprises essentially mannitol which is present in at least 25% by weight of the tablet.

2. A tablet as claimed in claim 1 adapted for administration to a paediatric patient.

3. A tablet as claimed in claim 1 in which the tablet is provided in a low dosage form which comprises from about 150 to 250 mg amoxycillin and a high dosage form which comprises about 350 to 450 mg amoxycillin.

4. A tablet as claimed in claim 1 in which amoxycillin is present in from 45 to 60% by weight of the tablet.

5. A tablet as claimed in claim 1 in which mannitol is present in from 30 to 50% by weight of the tablet.

6. A tablet as claimed in claim 1 which comprise further excipients, for instance, disintegrants, lubricants, sweetening agents, colouring and flavouring agents.

7. A tablet as claimed in claim 1 in which a disintegrant may be present in from 1 to 4% by weight of the tablet.

8. A tablet as claimed in claim 7 in which the disintegrant is crospovidone.

9. A tablet as claimed in claim 1 in which a lubricant may be present in from 0.25 to 2.0% by weight of the tablet

10. A tablet as claimed in claim 9 in which the lubricant is magnesium stearate.

11. A tablet as claimed in claim 1 in which the sweetening agent is an artificial sweetening agent.

12. A tablet as claimed in claim 11 in which the artificial sweetening agent is aspartame.

13. A tablet as claimed in claim 3 in which the low dosage tablet has a total weight in the range 400 to 500 mg and the high dosage tablet has a total weight in the range 850 to 950 mg.

14. A chewable tablet which comprises from 50 to 55% amoxycillin trihydrate; from 40 to 45% mannitol; from 1 to 2% disintegrant; from 0.5 to 1.2% lubricant; from 0.5 to 1.5% artificial sweetening agent; and flavouring and colouring agents.

15. A method of treating bacterial infection in a paediatric patient which comprises administering to said patient an effective amount of a tablet as claimed in claim 1.

16. A method as claimed in claim 14 in which the dosage is administered every 12 h.

17. A pharmaceutical granule comprising amoxycillin trihydrate present in from 45 to 60% by weight of the granule and mannitol present in from 25% by weight of the granule.

18. A granule as claimed in claim 18 further comprises a disintegrant present in from 1 to 4% by weight of the granule.

19. A granule as claimed in claim 18 which further comprises a lubricant present in 0.125 to 1.0% by weight of the granule.

20. A granule as claimed in claim 18 which comprises amoxycillin trihydrate present in from 50 to 55%, mannitol present in from 40 to 45%, crospovidone present in from 1 to 2%, and magnesium stearate present in from 0.25 to 0.6%; all %s being by weight of the granule.

21. A chewable tablet comprising granules comprising amoxycillin trihydrate present in from 45 to 60% by weight of the granule and mannitol present in from 25% by weight of the granule.

22. A chewable tablet as claimed in claim 20 which further comprises and an extra-granular lubricant, present in about 50% of the total amount of lubricant in the tablet.
